(19) United States
(12) Patent Application Publication
(10) Pub. No.: US 2015/0379362 A1
(43) Pub. Date: Dec. 31, 2015

(54) IMAGING DEVICE BASED OCCUPANT MONITORING SYSTEM SUPPORTING MULTIPLE FUNCTIONS

(71) Applicant: IEE INTERNATIONAL ELECTRONICS & ENGINEERING S.A., Echternach (LU)

(72) Inventors: Sam CALMES, Luxembourg (LU); Cecile JOUANIQUE-DUBUIS, Evrange (FR); Thierry MOUSEL, Mondorf (LU); Jean-Luc KAISER, Erpeldange (LU); Laurent LAMESCH, Reichlange (LU); Bruno MIRBACH, Konz (DE)

(21) Appl. No.: 14/769,320

(22) PCT Filed: Feb. 21, 2014
(86) PCT No.: PCT/EP2014/053472

§ 371 Date: Aug. 20, 2015

(30) Foreign Application Priority Data
Feb. 21, 2013 (LU) LU 92 158
Dec. 9, 2013 (LU) LU 92 329

(51) Int. Cl. G06K 9/00; G06K 9/46; H04N 5/76; A61B 5/18; G06T 7/20; A61B 5/024; A61B 5/00; G01B 11/25; H04N 5/243

(52) U.S. Cl. CPC G06K 9/00838; G01B 11/254; G06K 9/46; G06K 9/4661; H04N 5/76; H04N 5/243; G06T 7/20; A61B 5/02427; A61B 5/6893; A61B 5/18; G06K 2009/4666

(57) ABSTRACT

One or more imaging device(s) inside a car that look(s) at occupants (driver, front passenger, rear passengers) and cover(s) multiple security, comfort, driver assistance and occupant state related functions, wherein the imaging device includes an illumination in the near infrared. An imaging device inside the car that can measure occupants' vital signs (heart rate, respiration rate, blood oxygen saturation) using contactless imaging photoplethysmography.

### TECHNICAL FIELD

The present invention generally relates to a monitoring system for monitoring occupants in a closed environment. The present invention more particularly relates to an occupant monitoring system for automotive vehicles based on at least one imaging device. In a preferred application, the invention relates to a vehicle interior imaging device to perform a number of combined functions covering safety, driver assistance, comfort and occupant state. Contactless measurement of vital signs (heart rate, breathing rate and blood oxygen saturation) using an imaging device.

### BACKGROUND ART

Current occupant monitoring systems embedded into automotive vehicles are mainly dedicated to the occupancy detection function through seat-located sensors. These monitoring systems usually comprise some kind of seat occupancy detector mounted in the seat for detecting, whether the seat is occupied. Those systems cannot consistently differentiate between occupants and objects.

In parallel, a few systems for driver state assessment are emerging inside the car: these systems are using either remote 2D cameras or contact photoplethysmography or try to measure driver performance via steering angle or lane keeping.

The need for human-selective seat occupancy detection and for driver's state monitoring in general and driver's vital signs monitoring in particular increases with the penetration of the advanced driver assistance systems, like emergency braking, lane keeping and e-call systems, which may be enhanced by taking into account inputs from the driver state and behavior. One solution for this need has been disclosed in the international patent application WO 2013/020648 A1. This documents discloses the use of imaging photoplethysmography (iPPG), where an imaging sensor is used to measure reflectivity changes due to blood volume changes in the skin, in order to monitor the vital signs of one or more vehicle occupants.

The disclosed device can measure vital signs (heart rate, breathing rate, oxygen saturation) using contactless imaging photoplethysmography on exposed areas of the skin (typically the head) This is better than alternative methods which are involving contact methods (ECG, EEG) where the driver needs to wear electrodes or put both hands in certain positions on the steering wheel. It is also preferred to capacitively measured ECG (cECG) as here multiple electrodes need to be integrated into the car seat (cost, complexity, different for each car seat type) and potentially more reliable (cECG is sensitive to clothing thickness and type, electrode placement, motion artifacts, sweating).

One disadvantage of the iPPG device however lies in the fact that the measurement principle requires the imaging of exposed areas of skin. Accordingly monitoring by iPPG is not possible where no exposed skin is visible by the detector. This may e.g. be the case for small children, which are strapped into auxiliary child seats and which are covered for instance by a blanket or the like. Accordingly the iPPG system may not reliably detect sleeping children or babies left intentionally or unintentionally in the car.

### BRIEF SUMMARY

The disclosure provides an improved occupant monitoring system.

An automotive vehicle occupant monitoring device comprises at least one source of electromagnetic radiation, e.g. visible or infrared light, preferably in the near infrared, said source of electromagnetic radiation for generating electromagnetic radiation and for projecting said electromagnetic radiation in a projected pattern into a region of interest within an interior compartment of said automotive vehicle. At least one imaging device is used for detecting reflected radiation of said projected pattern, said scattered radiation being reflected or scattered from one or more objects located within said region of interest (specular or diffuse reflection). According to the invention, a detection unit is operatively coupled to the at least one imaging device, said detection unit comprising an intensity evaluation module for evaluating an intensity or amplitude of said reflected radiation over time.

By monitoring the intensity or amplitude of the reflected light, it is possible to detect slight variations of the amplitude or intensity of the reflected light and accordingly to detect a variation of the distance between the imager and the scattering or reflecting object. The occupant monitoring system thus enables to detect the respiratory movement of an occupant, e.g. of the thorax of the occupant. This detection can be performed on the occupants clothing or on a blanket covering an infant as in contrast to iPPG the detection of the respiratory movement does not require the visibility of exposed skin. The occupant monitoring system of the present invention thus enables a reliable detection of some vital signs and thus the presence of an occupant.

It will be noted that the above described measurement principle is particularly enabled by an active point source illumination which results in a radial intensity distribution that is inversely proportional to the square of the distance to the camera. Accordingly said projected pattern comprises preferably one or more radiation spots.

In a possible embodiment of the invention the source of electromagnetic radiation comprises a controllable projecting unit configured for projecting the projected pattern to a plurality of defined positions within said region of interest. The detection unit operatively is then operatively coupled to said controllable projecting unit and configured for controlling the position of the projected pattern and for evaluating an intensity or amplitude of said reflected radiation over time from said plurality of defined positions. Such a solution increases the flexibility of the monitoring device and enables an occupant monitoring a different locations within the vehicle compartment.

The occupant monitoring system according to the invention may be configured for the combined monitoring using different detection methods. In one preferred embodiment for instance, the detection unit is further configured for performing imaging photoplethysmography (iPPG) on the basis of the reflected radiation. Alternatively or additionally, the imaging device may be configured for recording situational images of the region of interest in which case said detection unit is further configured for optical pattern recognition in the recorded situational images. By combining physical measurements (e.g. looking at eyelid closure, head movements and facial expressions) and physiological measurements (heart rate and heart rate variability, breathing rate) results in a more robust device for assessing sleepiness

The automotive vehicle occupant monitoring device may additionally be provided with light compensation means for compensating the influence of changing ambient light conditions and/or motion compensation means for compensating the influence of motion of the object within the region of interest.

It will be appreciated that the present invention also relates to an automotive vehicle comprising at least one automotive vehicle occupant monitoring device as described here above. In such an automotive vehicle the region of interest preferably includes the front seat area and/or the rear seat area of a vehicle compartment.

The output signal of said occupant monitoring device may be used in one or more of robust occupant detection (while discriminating objects), seat belt reminder function, seat classification for airbag, child left behind detection, optimization of driver assistance systems, air conditioning optimization and automated emergency call support functions.

It is e.g. suggested to use a 2D interior imaging device that covers multiple functions such as:

a. Safety functions:
Drowsiness, sleepiness detection
Seat belt reminder
Unattended child detection/hyperthermia
Passenger seat classification for airbag and seatbelts
Alcohol and drug detection
Distracted driver detection
Heart attack detection
User differentiation system (UDS)
Seat belt early tension release for elderly people
Allowed driver, driver learner passenger detection

b. Advanced driver assistance systems support
Support of lane departure, automated breaking and stop and go functions

c. Comfort functions
Vehicle settings customization
Air conditioning optimization
Headrest height adjustment
Rearview and side view mirror adjustment
Adaptive seat position and belt height adjustment
Adaptive head-up display
Gesture recognition
Intrusion detection
Video conferencing

d. Occupant state detection (non-safety functions)
Emotions detection
Health checkup and health history
Automated emergency call support

Furthermore vital signs of the driver and also of the remaining occupants are measured by the imaging device using contactless imaging photoplethysmography. For this, the imaging device includes an infrared illumination so that it works independent of lighting conditions and in particular at night.

### BRIEF DESCRIPTION OF THE DRAWINGS

Further details and advantages of the present invention will be apparent from the following detailed description of several not limiting embodiments with reference to the attached drawings, wherein:

FIG. 1 is a schematic illustration of the components of an occupant monitoring device;

FIG. 2 is a diagram summarizing functions covered by car interior imaging device;

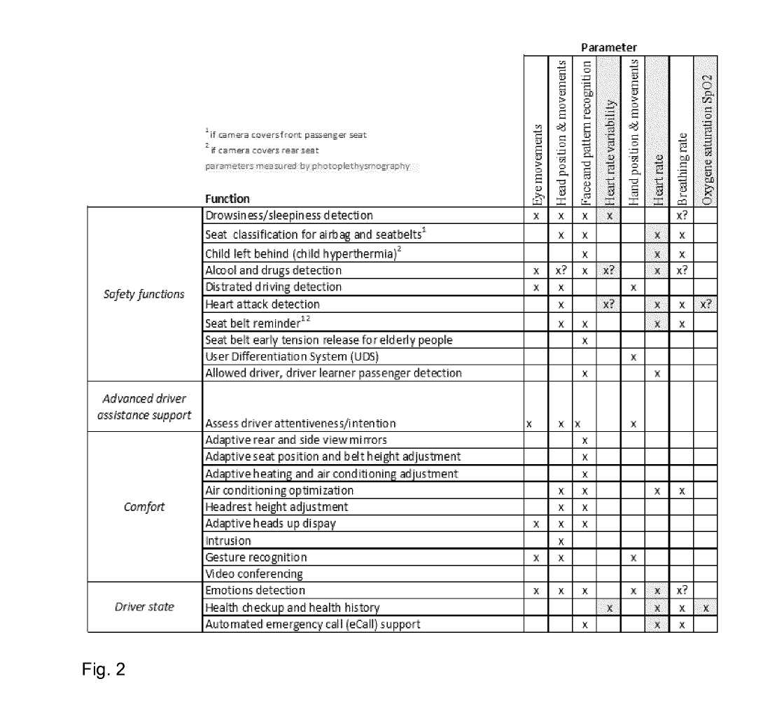

FIG. 3 is a schematic illustration of possible locations of a car interior imaging device.

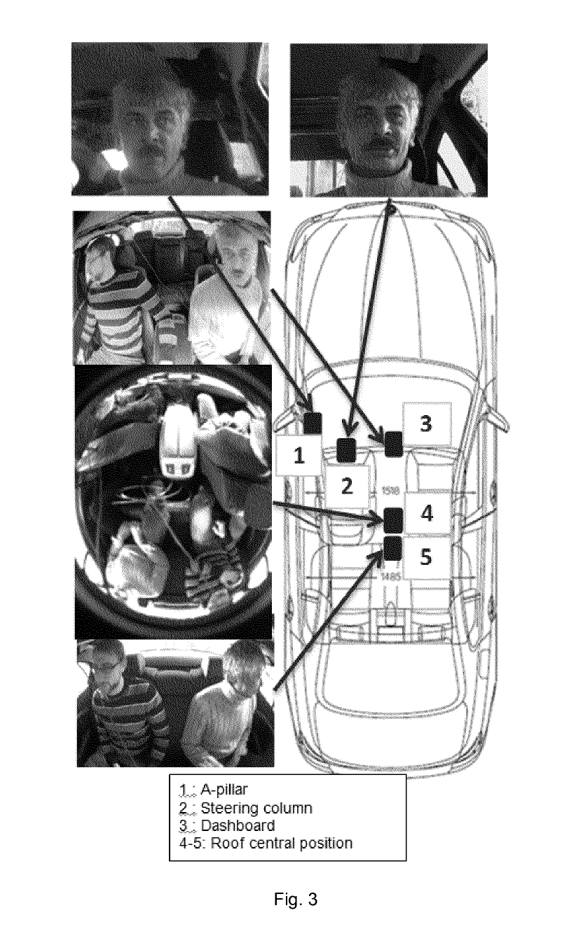

### DESCRIPTION OF PREFERRED EMBODIMENTS

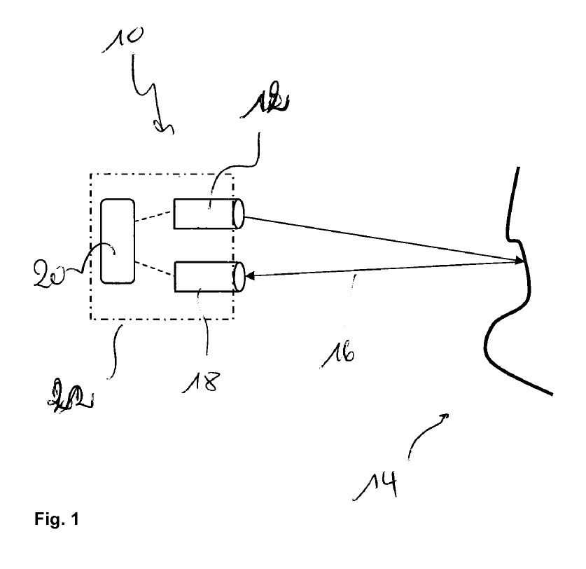

FIG. 1 shows a schematic illustration of the components of an occupant monitoring device 10. A illumination source 12 emits an active point illumination into a region of interest 14 where the light is reflected e.g. on the thorax of an occupant. The reflected light 16 is detected by an imaging device 18. A detection unit 20 operatively coupled to the imaging device 18 and the illumination source 12. The detection unit 20 comprises an intensity evaluation module for evaluating an intensity or amplitude of said reflected radiation over time.

By monitoring the intensity or amplitude of the reflected light, it is possible to detect slight variations of the amplitude or intensity of the reflected light 16 and accordingly to detect a variation of the distance between the imager 18 and the scattering or reflecting object. The occupant monitoring system 10 thus enables to detect the respiratory movement of an occupant, e.g. of the thorax of the occupant.

It will be noted that the illumination source 12 and the imaging device 18 may be integrated at different locations in a vehicle. In the preferred embodiment of FIG. 1, the illumination source 12, the imaging device 18 and the detection unit are arranged in a common housing 22.

One or more of the occupant monitoring devices 10 may be installed inside a car interior and looking at occupants (driver, front passenger, rear passengers) perform a number of useful functions that cover or support safety, advanced driver assistance, comfort and occupant state monitoring functions.

The imaging device can measure vital signs (heart rate, breathing rate, oxygen saturation) using contactless imaging photoplethysmography on exposed areas of the skin (typically the head) or by measuring slight variations of amplitude of the reflected light (typically the thorax area). The latter measurement principle is particularly enabled by an active point source illumination which results in a radial intensity distribution that is inversely proportional to the square of the distance to the camera. This is better than alternative methods which are involving contact methods (ECG, EEG) where the driver needs to wear electrodes or put both hands in certain positions on the steering wheel. It is also preferred to capacitively measured ECG (cECG) as here multiple electrodes need to be integrated into the car seat (cost, complexity, different for each car seat type) and potentially more reliable (cECG is sensitive to clothing thickness and type, electrode placement, motion artifacts, sweating).

Contactless EEG methods have also been investigated. Another way to measure physiological signals is by using mechanical vibration sensors such as ferroelectret films.

Such vital signs can be used to assess the driver's fatigue state (and thus warn him before he falls asleep), detect signs of impeding heart attack (and warn him) or detect the heart attack itself (and slow down and park the car, trigger an automatic emergency call (eCall)) and monitor his health/fitness level. Such vital signs can also be used to measure or reinforce the measurement of human presence in a car and distinguish from large objects.

One of the major challenges of imaging photoplethysmography or reflected light amplitude variation measurement in the car is that the ambient light conditions change a lot. One example is driving through a tree-lined alley during a sunny day where the car shade and sunlight will alternate fast. Another example is driving at night, where the car is illuminated by changing artificial light such as headlights from other cars passing by or street lights. These changing light conditions can be compensated by the following methods

a) Alternating active illumination
Preferably, the active illumination is switched on only for the recording of every second frame. The difference between a successive illuminated and non-illuminated frame is calculated, and this difference frame is then used for subsequent processing. This procedure substantially eliminates the influence of non-correlated background illumination.

b) Frequency close to power grid frequency
Additionally, the frame rate is preferably set substantially equal to submultiples of the power grid frequency employed in the region where the application is deployed, thereby eliminating correlated interference by artificial lightning, for example street lights.

c) Active illumination with adapted filter
The camera might contain an optical band-pass filter (BPF) in the receiving optical path together with illumination sources which have a small spectral bandwidth. With such a setup, one blocks as much as possible of the ambient light and transmits as much as possible of the active light. There is a direct correlation between the bandwidths of the BPF and the source and the SNR in changing ambient light conditions.

d) Reference signal where no persons
By measuring reflected light amplitudes in zones where it is known that no person is present one can compensate the background light on people.

e) Light modulation
By modulating the light source and using demodulation pixel architectures to distinguish between active and ambient light or using more than one wavelength in the illumination source together with a BPF having more than one adapted transmission window and using spatial or temporal multiplexing of these spectral bands.

Another challenge for imaging photoplethysmography or reflected light variation measurement in the car is that they are sensitive to motion of the subject under measurement. Several motion compensation techniques could be used.

a) Radial motion compensation
Radial motion of the person will lead to changing light amplitudes on the face of that person. This changing light can be compensated by using feature detection and tracking of the 2D camera (for example distance between eyes or head diameter) From the feature motion a scale change will be determined that allows to compensate the distance dependence of the light power density on the scene. Alternatively one can use 3D cameras (time of flight, modulated light intensity, stereoscopic).

b) Lateral motion compensation
Lateral motion of the person will lead to changing light conditions of the region being measured and different points being measured. By feature detection and tracking the point being measured can be tracked. If one knows the light distribution, the light variation of the region being tracked can be compensated.

The occupant monitoring device will allow to perform a more robust driver sleepiness or driver drowsiness detection. Sleepiness can be detected by 3 fundamental methods: 1) physical: look at eye movements, eyelid closure, head movements, facial expressions (yawning). 2) Look at physiological parameters: heart rate, breathing rate, heart rate variability (or HRV, which has been linked to sleepiness and is able to detect the onset of microsleep). 3) driver performance (steering wheel movements, ability to keep the lane). Current methods usually only use method 1) or 2) while sometimes combining with 3). This is usually not enough as a) the methods are not always reliable in all conditions and b) the methods might depend on particular behavior or might be triggered by certain persons more easily, leading to either false alarms or unreliable detection. Here we suggest to combine physical and physiological measurements using the same sensor (an imaging device) in order to assess both physical and physiological parameters, leading to a more robust assessment of driver sleepiness.

Current driver assistance systems, such as stop and go assistance, lane keeping assistance and emergency breaking systems do not take into account the driver attentiveness or driver intentions. This either leads to false warnings that can be perceived as annoying or ineffective driver assistance systems.

### Examples

The imaging device will be able to monitor head movements and eye gaze.
   a) Driver attentiveness: A potentially distracted driver (looking sideways, tuning the radio) can be alerted by e.g. a forward collision warning system in a more adequate way. Warning time, -intensity and -strategy can be adapted to the drivers attentiveness and his viewing direction (an attentive driver looking at the cars in front of him can be warned later to avoid unnecessary or too early alerts that might annoy the driver, while a distracted driver needs to be warned earlier).
   b) Driver intention: On a highway while driving on the slow lane and closing in on a car in front, the automated breaking system might not start breaking or might start breaking later if the imaging device has detected a gaze into the side view mirror in anticipation of a lane change.
   c) Driver intention: The lane keeping assistant is enabled on a highway without much traffic and the driver changes the lane intentionally but forgets to switch on the turn signal. In such a case, the lane keeping assistant issues a warning. This is often perceived as an annoyance and many drivers do not use the lane keeping assist function anymore. To avoid this, the imaging device could track the eye movements (track the driver's gaze) and if the driver changes lanes immediately after having looked into the side view mirrors, the lane assistant warning could be suppressed.
The imaging device is able to assess the driver's sleepiness.
   d) This knowledge can be used to adapt the driver assistance system's response as well (put them on ‘higher alert’/sensitivity).

Finally, there are liability issues associated with the new driver assistance systems for which the car manufacturers do not want to take full responsibility. For example, for the stop and go as well as the lane keeping assistant functions, drivers have the tendency to relax and remove their hands from the steering wheel, which can lead to dangerous situations. The imaging device could detect the position of the hands on the steering wheel and provide this information to the driver assistance system.

The imaging device will allow to perform a more robust occupant detection. In addition to determining presence of persons in a seat by using optical pattern recognition, the imaging device will be able to provide a more robust assessment of human presence (and distinguish from large objects) by determining for each object identified by optical pattern recognition a corresponding vital sign. This allows to realize intelligent seat belt reminder systems also on the rear seats, where conventional seat based occupant detection sensors (as used on the front seats) are less suitable because of: folding/removable seats, frequent transportation of objects, more “freedom of movement” for the occupants.

In particular, a combination between optical pattern recognition and vital signs determination (either by imaging photoplethysmography on exposed skin areas or by light amplitude variation measurements on the chest for example) performed by the same imaging device will allow to reliably detect sleeping children or babies left intentionally or unintentionally in the car. This could save an estimated few hundred lives worldwide every year where small children die because left unattended or forgotten in a car exposed to the sun.

An imaging device is thus proposed based on a standard two-dimensional imaging chip such as used in modern cameras. The imaging device can look at the driver, the front seat occupant or the rear seat occupants or a combination thereof. In order to see the interior car scene at all times and in particular at night, either a near infrared illumination, which is invisible to the human eye, is used, or alternatively the scene is illuminated by the car ambient lighting. In the latter case, an illumination color that presents absorption peaks for hemoglobin, such as green, should be used so that one gets the best photoplethysmographic signal such as to be able to measure vital signs.

With such a device, the following functions can be covered:

### Drowsiness/Sleepiness Detection

Driver drowsiness or sleepiness or fatigue is the cause of a large number of accidents (some sources relate up to 25% of all accidents to driver fatigue). The problem is exacerbated in monotonous driving conditions (such as highways) at night. People who experience microsleeps usually remain unaware of them. Needless to say that in a car such a situation is extremely dangerous. The challenge is to detect the sleepiness before microsleep occurs, so that the driver can be warned accordingly. Once sleep has occurred, its detection is still useful as the car could be slowed down and parked autonomously. Driver sleepiness can be detected by the following parameters, all measureable by an imaging device:

### a) Eye Movements

Sleepiness or onset of microsleep can be detected by tracking eyelid movement and percentage of eyelid closure (PERCLOS) [14,3,11]. These methods have shown to correlate with lapses in visual attention.

Eye gaze and pupil diameter can also be used to assess sleepiness [3].

These parameters can be measured using image processing techniques.

### b) Pupil Diameter

Changes of pupil dilation has been connected to cognitive workload or cognitive activity or cognitive effort. Keeping track of this parameter would allow to estimate how busy or cognitively loaded a driver is.

### c) Head Position and Movements

Head nodding can increase before the onset of microsleep [3]. Therefore tracking head position x, y, z can be an indicator of driver sleepiness [3,7]. The head movement forward and sideways can be tracked by image processing techniques.

### d) Facial Pattern Recognition

One can detect sleepiness by looking at certain facial patterns, such as yawning. Such facial patterns can be detected by optical pattern recognition algorithms.

### e) Vital Signs

Heart rate variability (HRV) has been linked to sleepiness. Heart rate and heart rate variability could be measured by using imaging photoplethysmography. Photoplethysmography is subject to motion induced artifacts, which need to be compensated by motion compensation algorithms. These algorithms correct for example planar shifts of the region of interest. In order to deal with varying light conditions an IR bandpass filter should be used, only letting the light from the near IR illumination through. Breathing rate can also be detected by measuring small light amplitude variations on the chest for example.

Other vital signs, such as heart rate and respiration rate, both measurable by imaging photoplethysmography, could also be used to assess driver sleepiness.

### Seat Belt Reminder

This function can apply to both the front passenger and the rear passengers. Objective is to detect the presence of a person and trigger a seat belt reminder if the person is not wearing the seat belt. If the seat is empty, or if there is an object on the seat there should be no seat belt reminder warning. The following parameters can be used to detect presence of a person or detect the seat belt directly (and potentially saving the current seat belt buckle switch, all measureable by a camera.

### a) Pattern Recognition and Moving Objects

An optical pattern recognition algorithm could determine the presence of a person in the front passenger seat or the number of persons present on the rear seats or rear bench. In addition to looking at patterns (shapes), the algorithms can look at movements in order to asses human presence.

Using the same optical pattern recognition techniques, the deployed seat belts can be detected directly by looking at the contrast between seat belt and underlying clothing.

### b) Vital Signs

A detection of a vital sign, such as a heart rate of breathing rate, detected via imaging photoplethysmography or in the case of breathing rate, via detection of minute movements of the chest in the frequency of interest by measuring light amplitude variations, could reinforce the distinction between person and large object provided by optical pattern recognition: for each object recognized as a person by optical pattern recognition, the imaging device can look for a vital sign on the object. If there is a vital sign present, the object identified by optical pattern recognition is for sure a person. Thus a very robust determination of human presence can be provided by a single imaging device.

For the rear seat, a single camera can look at the three rear seats or rear seat bench and determine multiple persons at the same time.

### Child Left Behind/Hyperthermia

The ‘child left behind’ function looks for a sleeping or non-sleeping child or baby left behind in the car. This is a very dangerous situation when the sun is shining as the temperatures can rise very fast inside a car and children (especially babies) are very sensitive to rising temperatures. See [http://ggweather.com/heat/]

### a) Pattern Recognition and Vital Signs

The same parameters as outlined under Seat belt reminder above can be used to determine if a child was left behind in a car.

### Seat Classification for Airbag and Seatbelts

Of interest here is the classification of the occupants into senior versus adult versus child, child seat, object and empty seat. In addition, knowing the position of the head is important for safe airbag deployment. This allows for smart airbag deployment (adapted force or suppression if no person present) and adequate seatbelt pretensioning in case of an accident. The head position is of interest to allow for a softer airbag deployment if the person is leaning forward or to automatically adjust the headrest height.

### a) Pattern Recognition, Face Recognition and Head Position

Optical pattern recognition algorithms could determine whether the seat is occupied, and if occupied, whether it is an adult, a child, a rear facing child seat, an object or an empty seat.

Optical pattern recognition algorithms could also determine the position of the head (proximity to the airbag) so that a softer airbag deployment can be used if the head is closer to the airbag before deployment.

In addition, the person size and age can be estimated using face feature recognition algorithms, allowing restraint system adaptation, e.g. for a ‘softer’ seat belt load limiter for elderly persons whose rib cage is less robust.

Finally, the person weight can be estimated using algorithms that look for body size as seen from the imaging device. This allows for an appropriate airbag deployment.

### b) Vital Signs

Similarly as explained under b) Vital signs, the detection of a vital sign can reinforce the decision from the pattern recognition algorithms in determining human presence.

### Alcohol and Drug Detection

### a) Eye Movements and Facial Patterns

The following physical parameters can be measured on a driver under the influence of alcohol:

Involuntary eye movements (Horizontal Gaze Nystagmus (HGN))

Eye and facial patterns

Pupil diameter and eye movement

These physical parameters can be tracked by optical pattern recognition using an imaging device.

### b) Spectrometry

One can measure alcohol by tissue spectroscopy where the skin is illuminated by the NIR light of the optical device illumination and the reflected light is analyzed to determine the alcohol concentration.

Similarly, one can measure alcohol by gas imaging spectroscopy where the air exhaled by the driver is illuminated by the NIR light of the optical device and the reflected light is analyzed to determine the alcohol concentration in the air.

### c) Heart Rate and Heart Rate Variability

Heart rate (HR) and heart rate variability (HRV) can be used to detect alcohol consumption. HR and HRV can be measured by imaging photoplethysmography.

### d) Breathing Rate

Breathing rate can be used to detect alcohol consumption. Using an imaging device, breathing rate can be detected either by imaging photoplethysmography or by measuring minute movements of the chest using image processing techniques in general and looking at reflected light amplitude variations in particular.

### Distracted Driver Detection

This function comprises detecting whether the driver is focused on the road. The following parameters can be used to detect distracted driving using a camera:

### a) Eye and Head Position

Determining the eye position, and particularly the location of the pupil, allows to determine where the driver is looking. Similarly, looking at the head position allows to determine where the driver is looking. If the driver is looking away from the road for too long, or if the driver is looking away from the road at a critical moment (for example determined by exterior cameras), appropriate action can be taken (warning signals, support of advanced driver assistance systems, pre-activation of safety systems).

### b) Hand Position and Movements

Hand positions and hand movements are an indicator of distracted driving and can be detected by a camera. If the hands leave the steering wheel (for too long or in a critical situation as assessed by other sensors), appropriate action can be taken.

Similarly, optical pattern recognition can be used to determine whether the driver is holding a handheld phone up against his ears by looking at patterns that look like a phone and hand position (history).

### Medical Emergencies Detection

NHTSA published in 2009 a study with the following conclusions:
“the percentage of drivers in crashes precipitated by their medical emergencies while driving are relatively rare and account for only 1.3 percent of all drivers that have been included in the study. Older drivers have relatively higher incidences of crashes precipitated by drivers' medical emergencies when compared to young and middle-age drivers.
crashes precipitated by drivers' medical emergencies are not related to vehicle design or roadway integrity as indicated by the type of crashes and manner of collisions. Patient education by health care providers on early warning signs of a health crisis, such as warning signs before seizure attacks, diabetic or hypoglycemic comas, and potential side effects of medications are recommended as the most effective countermeasure. In addition to patient education, other safety technologies such as the Drowsy Driver Warning System can help in reducing the risk of crashes precipitated by medical emergencies.”

### a) Head Position and Facial Patterns

Inappropriate head position, lasting over a quite long periods, combined to a rapid change in the facial expression, may indicate serious health impairment.

### b) Heart Rate

By looking at the heart rate or heart rate variability using imaging photoplethysmography one can detect or possibly anticipate medical emergency.

### c) Breathing Rate

Health crisis victims often show breathing irregularities which could be detected by a camera, using either photoplethysmography of detecting minute chest movements.

### User Differentiation System (UDS)

User differentiation system is a feature that blocks control of certain equipment, such as navigation system, on board TV and internet access to the driver while the vehicle is moving but leaves these functions available to the front passenger. The following camera parameters can be used to fulfill this function:

### a) Hand and Arm Position

The camera could track, via optical pattern recognition, the driver's respectively the front passengers hand and arm positions and lock certain equipment only if the driver tries to manipulate such equipment while driving.

### Driver Assistance System Support

Common driver assistance system functions are stop and go, lane keeping and automated breaking.

The stop and go functionality allows to accelerate and slow down the car in heavy traffic automatically by following the vehicle ahead.

The lane keeping assistant systems help the driver stay inside his lane by detecting the lane markings using forward looking cameras and by warning the driver or taking corrective measures (for example via steering wheel torque or ESC) if the vehicle leaves its lane and no reaction by the driver is detected.

Measuring driver attention would allow to adjust the driver assistance systems to the state of the driver. If the driver is alert or focused on the road for example, the systems need to assist less or warnings can be triggered later than when the driver is sleepy of distracted.

### a) Head Position and Eye Position

Pattern recognition algorithms that track eye gaze and head direction would allow to determine whether the driver is looking at the road ahead or not.

### b) Face and Pattern Recognition

One can detect sleepiness by looking at certain facial patterns, such as yawning. Such facial patterns can be detected by optical pattern recognition algorithms.

### c) Vital Signs

Heart rate variability (HRV) has been linked to sleepiness. Heart rate and heart rate variability could be measured by using imaging photoplethysmography. Photoplethysmography is subject to motion induced artifacts, which need to be compensated by motion compensation algorithms. These algorithms correct for example planar shifts of the region of interest. In order to deal with varying light conditions an IR bandpass filter should be used, only letting the light from the near IR illumination through. Breathing rate can also be detected by measuring small light amplitude variations on the chest for example.

Other vital signs, such as heart rate and respiration rate, both measurable by imaging photoplethysmography, could also be used to assess driver sleepiness.

### d) Hand and Arm Position

The camera could track, via optical pattern recognition, the driver's hand and arm positions before the stop and go function drives the car off from a stop or while the lane keeping assistant is enabled.

### Vehicle Settings Customization

Recognizing/identifying the driver or car occupant would allow to customize certain vehicle settings to their preference (which they have to set once). Such customization could include:
Rear and side view mirrors: adjust their position as a function of who is driving (person size)
Seat position: adjust the seat position (distance from steering wheel, car seat back tilt) depending on person size and driving position preference
Belt height: adjust belt height depending on person size
Heating and air conditioning: adjust ventilation, heating and cooling to the known preferences of recognized occupants

### a) Face Recognition

Face recognition algorithms allow to recognize a person and then change vehicle settings according to the known preferences of that person.

### b) Pattern Recognition

Pattern recognition algorithms allow to determine person seated height and provide a recommendation to unknown (not yet programmed) occupants for mirrors, seat position and belt height.

### Air Conditioning Optimization

The following parameters can be used to optimize the air conditioning in a car:

### a) Pattern Recognition

Assess the number and position of people inside the car using optical pattern algorithms and as a function of number of occupants and their position, adjust the ventilation power.

### b) Facial Features Recognition

Look at visible signs of discomfort on the face and adjust ventilation accordingly. For example, adjust temperature/air flow if an occupant shows signs of feeling too warm (e.g. sweating, red face). Recognize clothing (for example hat) and reduce the temperature accordingly.

### c) Heart Rate and Breathing Rate

Adjust temperature/air flow if a person shows signs of feeling too warm (linked to an increasing heart and/or respiration rate measure by imaging photoplethysmography).

### Headrest Height Adjustment

Electrical headrests can be moved to their lower position if the seats are not occupied by people. In addition, the headrest can be adjusted to a height that fits the occupant's size.

### a) Head Position

Optical pattern recognition algorithms can detect an occupant's head position respectively an empty seat, which allows to adjust the headrest height.

### Adaptive Head-Up Display

Head-up displays will become more common in tomorrow's cars. They can display relevant driving information in front of the driver without that he has to move his eyes from the road. They can also indicate danger situations and ways/directions to escape such dangerous situations.

In order to be most effective, the projection should happen exactly in front of the driver's eyes. Therefore it is important to know the driver's eye position and gaze direction.

### a) Eye Gaze and Head Tilt

Determining eye gaze and head tilt (using image processing) allows to display the head-up information on the right spot resp. allows to display different information depending on where the driver looks.

Eye gaze detection could allow to steer the user in a certain direction (e.g. bring his attention to a danger).

Optical pattern recognition algorithms could track eye gaze and head tilt.

### b) Head Position

By determining the head position (especially height), the head up display can be projected at the correct height, i.e. in front of the driver. Optical pattern recognition algorithms could track eye gaze and head tilt.

### Gesture Recognition

Gestures (head gestures, facial gestures or hand gestures) can be used to interact with the car and to perform certain commands in a vehicle. Thus the imaging device could act as human-machine interface (HMI).

### a) Pattern Recognition

Image processing and facial features detection techniques can be used to determine hand, arm, head or facial gestures, such as shaking head, nodding head, finger pointing.

### Emotions Detection

Detecting driver's emotions, for example irritation, could be used to get the driver out of an excessive emotional state (by proposing calming music or directing incoming calls to voicemail to an angry driver) or by proposing driving assistance or by making driving assistance more sensitive (put it on “high alert”) in such a situation.

### a) Eye Movements and Head Movements and Hand Movements

Body movements, such as excessive movements of eyes, head and hands can be an indication of emotivity. The following emotion related parameters can be measured using an imaging device:

### b) Facial Expressions

An imaging device could detect certain emotions by optical pattern matching with certain typical facial expressions of emotion.

### c) Heart Rate and Breathing Rate

Certain emotions, such disgust, happiness and surprise have been found to be accompanied by a low heart rate activity. Other emotions such as anger, fear and sadness have been found to be accompanied by a high heart rate (measured by imaging photoplethysmography).

Similarly, breathing rate patterns could be used to detect certain emotions (measured by reflected light amplitude variations).

### Health Checkup and Health History

The car is an environment where people spend a considerable amount of time in a rather calm position. Often they drive the same routes every day so one can record data under repeating conditions. It could be useful to measure the occupant's health or fitness for several purposes:
To follow a medical condition over time. Data could be analyzed locally by onboard computers or remotely by medical experts.
To provide real time feedback on physiological parameters or on general ‘fitness’ to the car occupants. For this the historic data can be used to provide a comparative assessment.
To link with medical services

### a) Heart Rate and Heart Rate Variability

Measured by imaging photoplethysmography, heart rate and heart rate variability are prime physiological health indicators. Recording and monitoring heart rate is of importance for many medical conditions, including of course heart disease.

### b) Oxygen Saturation

Oxygen saturation (SpO2), measured by imaging photoplethysmography, allows to determine oxygenation of blood. A normal oxygen saturation level is between 95% to 100%. Low oxygen saturation levels can be due to a number of different medical conditions, such as: blood oxygen transportation dysfunction (Anemia), air way obstruction, alveoli destruction. For example one could measure SpO2 to monitor occupants with asthma and warn if certain dangerous levels are crossed.

### Automated Emergency Call Support

Automated emergency call systems are designed to contact emergency services automatically in case of a severe accident. A camera could allow to provide the following information to emergency personnel:

### a) Pattern Recognition

Optical pattern recognition algorithms (coupled with vital sign information provided via PPG) allows to determine the exact number of occupants.

### b) Face Recognition

Face recognition algorithms allow to determine who is in the car. This allows to send crucial pre-programmed information out to emergency personnel such as blood type, medical history, medications taken etc.

### c) Vital Signs

Heart rate, breathing rate and blood oxygen saturation, all determined by imaging photoplethysmography, can be sent out in real time to emergency personnel so they know the condition of the occupants before reaching the scene.

### d) Picture or Movie Feed

A picture or movie feed of the situation inside the car could be taken after an accident so that emergency personnel can better assess the situation when organizing the emergency response

### Allowed Driver Detection

Face recognition algorithms allow to recognize the driver which allows to decide whether a person is allowed to drive a car. Car theft or carjacking or unhallowed use (by kids for example) can thus be prevented.

### Driver Learner Detection

Face recognition algorithms allow to recognize the passenger which allows to make sure that a) the driver learner is not driving the car alone and b) the driver learner is accompanied by an authorized person.

### Intrusion Detection

An imaging device, via pattern recognition algorithms, can detect an intrusion into a car and act as an alarm giver preventing theft.

### Video Conferencing

The camera could provide live video feed of car occupants for video conferencing with the outside world.

1. An automotive vehicle occupant monitoring device, comprising:
at least one source of electromagnetic radiation, said source of electromagnetic radiation for generating electromagnetic radiation and for projecting said electromagnetic radiation in a projected pattern into a region of interest within an interior compartment of said automotive vehicle,
at least one imaging device for detecting reflected radiation of said projected pattern, said scattered radiation being reflected or scattered from one or more objects located within said region of interest; and
a detection unit operatively coupled to said at least one imaging device, said detection unit comprising an intensity evaluation module for evaluating an intensity or amplitude of said reflected radiation over time.

2. The automotive vehicle occupant monitoring device according to claim 1, wherein said projected pattern comprises one or more radiation spots.

3. The automotive vehicle occupant monitoring device according to claim 1, wherein said source of electromagnetic radiation comprises a controllable projecting unit configured for projecting the projected pattern to a plurality of defined positions within said region of interest, and wherein said detection unit is operatively coupled to said controllable projecting unit and configured for controlling the position of the projected pattern and for evaluating an intensity or amplitude of said reflected radiation over time from said plurality of defined positions.

4. The automotive vehicle occupant monitoring device according to claim 1, wherein said electromagnetic radiation is an infrared light.

5. The automotive vehicle occupant monitoring device according to claim 1, wherein said detection unit is further configured for performing imaging photoplethysmography (iPPG) on the basis of the reflected radiation.

6. The automotive vehicle occupant monitoring device according to claim 1, wherein said imaging device is configured for recording situational images of the region of interest and wherein said detection unit is further configured for optical pattern recognition in the recorded situational images.

7. The automotive vehicle occupant monitoring device according to claim 1, further comprising light compensation means for compensating the influence of changing ambient light conditions.

8. The automotive vehicle occupant monitoring device according to claim 1, further comprising motion compensation means for compensating the influence of motion of the object within the region of interest.

9. Automotive vehicle comprising at least one automotive vehicle occupant monitoring device according to claim 1.

10. Automotive vehicle according to claim 9, wherein said region of interest includes a front seat area and/or a rear seat area of a vehicle compartment.

11. Automotive vehicle according to claim 9, wherein an output signal of said occupant monitoring device is used in one or more of robust occupant detection while discriminating objects, seat belt reminder function, seat classification for airbag, child left behind detection, optimization of driver assistance systems, air conditioning optimization and automated emergency call support functions.
